(19) United States
(12) Patent Application Publication
(10) Pub. No.: US 2009/0035298 A1
(43) Pub. Date: Feb. 5, 2009

(54) METHODS TO TREAT ALZHEIMER'S DISEASE OR OTHER AMYLOID BETA ACCUMULATION ASSOCIATED DISORDERS

(75) Inventors: David Holtzman, St. Louis, MO (US); Jae-Eun Kang, St. Louis, MO (US); John Cirrito, St. Louis, MO (US); John Csernansky, Chicago, IL (US); Hongxin Dong, St. Louis, MO (US)

Correspondence Address: POLSINELLI SHALTON FLANIGAN SUELTHAUS PC, 100 SOUTH FOURTH STREET, SUITE 100, SAINT LOUIS, MO, 63102-1825, US

(73) Assignee: WASHINGTON UNIVERSITY IN ST. LOUIS, St. Louis, MO (US)

(21) Appl. No.: 12/108,065

(22) Filed: Apr. 23, 2008

Related U.S. Application Data
(60) Provisional application No. 60/913,419, filed on Apr. 23, 2007.

(51) Int. Cl. A61K 39/395; G01N 33/00; A61K 31/7088; A61K 38/16; A61K 38/00

(52) U.S. Cl. 424/130.1; 435/7.92; 514/44; 514/12; 514/2

(57) ABSTRACT

The present invention provides methods for treating amyloid-beta accumulation-associated disorders, such as Alzheimer's disease. The methods comprise modulating the concentration of amyloid-beta in the brain interstitial fluid. In particular, the methods comprise modulating the activity of corticotrophin-releasing factor (CRF), which in turn modulates the concentration of amyloid-beta.

### CROSS REFERENCE TO RELATED APPLICATIONS

This application claims the priority of U.S. provisional application No. 60/913,419, filed Apr. 23, 2007, hereby incorporated by reference in its entirety.

### GOVERNMENTAL RIGHTS

This invention was made with government support under grant number RO1-AG025824 from the National Institutes of Health. The government has certain rights in the invention.

### FIELD OF THE INVENTION

The present invention relates to methods for treating Alzheimer's disease and related disorders by modulating the activity of corticotrophin-releasing factor (CRF) and, consequently, reducing the concentration of amyloid-beta.

### BACKGROUND OF THE INVENTION

Alzheimer's disease (AD) is the most common cause of dementia and is an increasing public health problem. It is currently estimated to afflict 5 million people in the United States, with an expected increase to 13 million by the year 2050. Alzheimer's Disease leads to loss of memory, cognitive function, and ultimately loss of independence. It takes a heavy personal and financial toll on the patient and the family. Because of the severity and increasing prevalence of the disease in the population, it is urgent that better treatments be developed.

Biochemical, genetic, and animal model evidence implicates amyloid-beta (Aβ) as a pathogenic peptide in AD. The neuropathologic and neurochemical hallmarks of AD include synaptic loss and selective neuronal death, a decrease in certain neurotransmitters, and the presence of abnormal proteinaceous deposits within neurons (e.g., neurofibrillary tangles) and in the extracellular space (e.g., cerebrovascular, diffuse, and neuritic plaques). The main constituent of plaques is Aβ, a 38-43 amino acid peptide cleaved from the amyloid precursor protein (APP). Throughout life, soluble Aβ is secreted primarily by neurons, but also by other types of cells. Multiple lines of evidence suggest that Aβ accumulation and changes in its comformation to forms with high β-sheet structure are central in AD pathogenesis. In late-onset AD, the total amount of Aβ that accumulates in neural tissue is about 100-200-fold higher in AD brains versus control brains. Accumulation of Aβ first occurs in specific regions of the neocortex, including parts of the frontal, temporal, and parietal lobes, and the hippocampus, i.e., areas that have the earliest and most severe neuropathology in AD. Damage to these areas is manifested by the first clinical symptoms of AD, i.e., memory deficits and cognitive losses.

Despite recent advances in understanding the pathogenesis of the disease, there are few, if any, effective therapeutic treatments for AD. Current treatments address only the cognitive manifestations of the disease. What is needed, however, are treatment regimes that slow the progression and/or delay or prevent the onset of the disease.

### SUMMARY OF THE INVENTION

Among the various aspects of the invention is a first aspect that provides a method for modulating the concentration of amyloid-beta in the brain interstitial fluid of a subject. The method comprises modulating corticotrophin-releasing factor (CRF) activity in the subject, wherein CRF activity modulates the concentration of amyloid-beta.

Another aspect of the invention encompasses method for treating Alzheimer's disease in a subject. The method comprises administering a corticotrophin-releasing factor (CRF) regulator to the subject, wherein the CRF regulator decreases the concentration of amyloid-beta in the brain interstitial fluid of the subject.

A further aspect of the invention providers a method for decreasing the concentration of amyloid-beta in the brain interstitial fluid of a subject. The method comprises inhibiting corticotrophin-releasing factor (CRF) activity in the subject.

Other aspects and iterations of the invention are described more thoroughly below.

### BRIEF DESCRIPTION OF THE FIGURES

FIG. 1 demonstrates the effect of isolation stress on soluble amyloid-β (Aβ) levels within the hippocampus. The effects of 3 months of isolation stress on soluble Aβ levels within the interstitial fluid (ISF), tissue lysates, and amyloid precursor protein (APP) fragments in the hippocampus were analyzed. FIG. 1A illustrates that three months of isolation stress increased ISF Aβ levels to 184±23% of control levels in 4 month old Tg2576 mice (p=0.0006; n=10 per each group). In vivo concentrations of ISF Aβ in the hippocampus were 5309±145.0 and 2881±61.0 pg/ml in mice exposed to 3 months of isolation and control conditions, respectively. FIG. 1B and FIG. 1C show that both Aβ₄₀ and Aβ₄₂, as determined by ELISA, were elevated by 37.9±4.4% and 57.7±9.4%, respectively, in the carbonate soluble fraction of hippocampal lysates from mice after 3 months of isolation stress compared to controls (p=0.02; n=7-8 per each group). FIG. 1D depicts representative lanes from Western blots for full length APP (FL-APP), APP α-CTF, and APP β-CTF in hippocampal tissue under the two conditions (n=7-8 per group). FIG. 1E illustrates that the levels of FL-APP, α-CTF and β-CTF were not changed after 3 months of isolation stress compared to controls. Each band was normalized to the amount of α-tubulin in each lane. Data represent mean±standard error of the mean (SEM).

FIG. 2 demonstrates the effect of restraint stress on soluble Aβ levels within the hippocampus. The effects of 3 hours of restraint stress on soluble Aβ levels within the ISF and tissue lysates, and APP fragments in the hippocampus were analyzed. FIG. 2A shows that three hours of acute restraint stress increased ISF Aβ levels to 132±6.9% of baseline by 13 hr after the beginning of stress initiation in 3-4 month old Tg2576 mice (p=0.003; n=10 per each group). FIG. 2B and FIG. 2C show that there were no significant differences in the levels of either Aβ₄₀ or Aβ₄₂, in stressed versus control mice in the carbonate soluble fraction of the hippocampal lysates as measured by ELISA (n=8 per each group). FIG. 2D depicts representative lanes from Western blots for FL-APP, α-CTF and β-CTF in hippocampal tissue. The levels of FL-APP and β-CTF were not different between groups. FIG. 2E shows that the levels of α-CTF were significantly decreased by 17.23±3.404% in Tg2576 mice with 3 hour restraint stress compared to controls (p=0.0005; n=8 per each group). Each band was normalized to the amount of α-tubulin in each lane. Data represent mean±SEM.

FIG. 3 demonstrates the effect of corticosterone on hippocampal Aβ levels. Systemic administration with corticosterone (CORT) did not acutely alter ISF Aβ levels. The effects of high dose CORT on hippocampal ISF Aβ levels in 3-4 months old Tg2576 mice were analyzed. After the basal ISF Aβ levels were obtained for 10 hours, animals received an intra-peritoneal injection with 50 mg/kg of CORT. An equal volume of vehicle solution (100 μl of 15% of 2-hydroxypropyl-β-cyclodextrin; HPB in water) was used for control animals. There was no difference in ISF Aβ levels in CORT treated versus vehicle treated mice (n=8 per each group).

FIG. 4 demonstrates the effects of corticotropin releasing factor (CRF) on ISF Aβ levels. To examine the effect of CRF on hippocampal ISF Aβ levels, 100 and 200 nM of CRF were administrated by reverse microdialysis in the hippocampus of 3-4 month old Tg2576 mice. FIG. 4A shows that 100 nM CRF in the microdialysis fluid resulted in an increase of ISF Aβ levels 3 hours after drug infusion, whereas 200 nM CRF increased ISF Aβ levels immediately after drug infusion (n=5 per each group). Both 100 and 200 nM CRF increased ISF Aβ levels in a dose-dependent manner, reaching 138.3±7.027% and 171.9±17.83% of baseline by 12 hours, respectively (FIG. 4B; p<0.0001 and p=0.0001, respectively). Three hours of restraint stress increased ISF Aβ levels to 132±6.896% compared to baseline by 13 hours after the beginning of stress initiation (FIG. 4C; p=0.003; n=10 for stress). Treatment with α-helical CRF₉₋₄₁ (αCRF₉₋₄₁), a CRF receptor antagonist, given from 30 minutes prior to restraint stress until the end of the experiment, blocked the stress-induced increase in ISF Aβ levels (p=0.006; n=5 for stress+αCRF₉₋₄₁).

FIG. 5 demonstrates that neuronal/synaptic activity is involved in the stress-induced increase in ISF Aβ levels. Infusion with 5 μM of tetradotoxin (TTX) in the hippocampus, by reverse microdialysis, immediately decreased ISF Aβ levels reaching 58.5% of baseline by 17 hours from drug treatment in 3-4 month old Tg2576 mice. Three hours of restraint stress was given to mice 8 hours after TTX treatment. This resulted in no significant change in ISF Aβ levels compared to TTX-alone treated controls (n=5 per each group).

### DETAILED DESCRIPTION OF THE PREFERRED EMBODIMENTS

It has been discovered, as illustrated in the examples, that modulating corticotropin-releasing factor (CRF) activity results in the modulation of amyloid-β (Aβ) levels, which are elevated in neurodegenerative diseases such as Alzheimer's disease (AD). In particular, it has been discovered that repression of CRF function will decrease Aβ concentration in the interstitial fluid of the brain. The present invention, accordingly, includes compositions and methods for modulating Aβ concentration by modulating CRF levels or activity. Since increased Aβ concentration in the brain contributes to AD or other disorders mediated by Aβ accumulation, the discoveries provide new treatment strategies for these diseases and disorders.

### (I) Methods

One aspect of the present invention encompasses methods to treat, prevent, or delay AD or other disorders mediated by Aβ accumulation in a subject. The methods may be utilized to treat a subject that is at risk of developing AD or to treat a subject that already has any of the indications of AD. Similarly, the method may be utilized to treat a subject that is at risk of developing any indication of another disorder mediated by Aβ accumulation or to treat a subject that already has any of the indications of an Aβ accumulation associated disorder. Exemplary Aβ accumulation associated disorders include, but are not limited to AD, cerebral amyloid angiopathy (CAA), Down syndrome, and Lewy body dementia.

An embodiment of the present invention includes a method for modulating the concentration of Aβ in the brain interstitial fluid of a subject by modulating CRF activity in the subject. In an additional embodiment, the invention includes a method of treating Alzheimer's disease in a subject by decreasing the concentration of Aβ in the brain interstitial fluid by modulating CRF activity. Further, an embodiment of the invention provides a method of decreasing the concentration of Aβ in the brain interstitial fluid of a subject by inhibiting CRF activity.

The methods of the embodiments include modulating CRF activity by altering a CRF protein production step. Suitable steps include those necessary for the production of a CRF protein from a nucleic acid sequence such as CRF transcription, CRF translation, and CRF protein activity.

CRF activity can be modulated by treating a subject with an effective amount of a CRF regulator. The CRF regulator may be an antagonist or agonist that results in decreased or increased CRF activity, respectively. Suitable CRF regulators include, but are not limited to, antibodies, peptides, proteins, small molecules, oligonucleotides, RNA antisense, DNA antisense, or a combination thereof. An effective amount refers to the amount of CRF regulator that is sufficient so that Aβ levels are modulated. Aβ levels can be measured by the methods described in the Examples herein and by methods commonly known in the art.

Another aspect of the present invention includes a method of modulating CRF activity by altering CRF association with at least one CRF receptor, which includes CRF-R1, CRF-R2α, CRF-R2β, and CRF-R2γ.

### (II) Compositions

CRF is a 41 amino acid peptide that is secreted by the hypothalamus in response to stress and typically stimulates the release of adrenocorticotropic hormone (ACTH) from the anterior pituitary. ACTH generally binds to receptors in the adrenal cortex and activates the release of glucocorticoid hormones. In addition to the hypothalamus, CRF and its receptors are expressed in a variety of other locations in the CNS where it may act as a neuropeptide to modulate neuronal activity and signaling. In response to stress, CRF is released and ultimately leads to an increase in Aβ concentration. Thus, increasing CRF activity correlates with increasing levels of Aβ. Typically, the effects of CRF activity are mediated by CRF receptors 1 and 2, although CRFR1, in particular, appears to modulate stress-mediated effects of CRF in the hippocampus. CRF receptors are G-protein coupled and their stimulation results in activation of adenylate cyclase and protein kinase A. The CRF receptors include CRF-R1, CRF-R2α, CRF-R2β, and CRF-R2γ.

The present invention is directed to compositions that resultantly decrease CRF activity and ultimately decrease Aβ levels. As such, the invention contemplates CRF regulators, such as antagonists and agonists that alter CRF protein activity either directly or indirectly. Indirect modulation of CRF protein activity may occur at any step of CRF protein production including at the nucleic acid level, transcriptional level, translational level, or posttranslational level. Suitable CRF antagonists typically prevent or reduce CRF activity, while CRF agonists induce or increase CRF activity.

### Regulation of CRF Protein Production

At the transcriptional level, CRF mediation of Aβ levels may be modulated by manipulating regulators of CRF transcription or mRNA production from the CRF-encoding DNA. For instance, transcriptional regulators that activate, or enhance, CRF transcription may be inhibited by methods described herein, to block CRF transcription and subsequent protein production resulting in lower CRF levels. In contrast, transcriptional regulators that repress, or inhibit, CRF transcription may be exogenously introduced to block CRF transcription and subsequent protein production resulting in lower CRF levels. Methods for exogenously introducing transcription regulators into a system include introduction by way of expression vectors, gene therapy, and other methods known in the art. Further, mRNA production may be modulated by using phosphorothioate oligonucleotide, 2′-O alkyl oligonucleotide, peptide nucleic acid, or locked nucleic acid antisense specific for the CRF transcript.

At the translational level, CRF mediation of Aβ levels may be inhibited by manipulating CRF translation. Exemplary methods of manipulating CRF translation include those using small RNAs (e.g., siRNA, shRNA, miRNA. etc.) for RNA interference, morpholino antisense probes specific for CRF mRNA, as well as other methods known in the art.

At the protein activity level, mediation of Aβ levels may be modulated by manipulating CRF receptors, binding to CRF protein by a means that disrupts normal protein activity (i.e., steric hindrance), or other means known in the art that results in reduced or increased CRF protein activity. For instance, suitable antagonists active at the protein level include, but are not limited to, peptides, proteins, small molecules, and antibodies that interact with CRF or CRF receptors that block CRF activity. By way of example, any peptide of at least 10, preferably 20, 25, 30, 35, 40, 50 or more amino acids of the CRF coding sequence, or any fragment of a sequence thereof, may be used to raise antibodies, derive peptides, or derive small molecules suitable for modulating the interaction between CRF with its natural receptors or CRF protein activity. Further, an inhibitory peptide or protein may comprise portions of the CRF protein necessary for binding to CRF receptors, but not include portions that are necessary to activate the receptor (dominate-negative peptides or proteins), therefore competing with endogenous CRF protein for receptor binding and reducing CRF mediated increase of Aβ levels.

### CRF Regulatory Small Molecules

The invention contemplates small organic molecules or any other compounds that may be designed through rational drug design, as known in the art, to modulate CRF protein activity. Suitable small molecules may be either peptidic or nonpeptidic. For example, small molecule inhibitors may be isolated using techniques known to those skilled in the art, such as high-throughput screening of chemical libraries, protein-protein binding assays, biochemical screening assays, immunoassays and cell based assays. Exemplary small molecule inhibitors that act as CRF receptor antagonists include those described in WO94/13676, WO95/10506, WO98/42699, JP11335373-A, JP2000063277-A, JP2000063378-A, as well as CRF receptor antagonists known as CP154, 526 (Schultz, D. W. et al, PNAS USA 93:10477, 1996), CRA1000 (Chaki, S. et al., Eur. J. Pharmacol., 371, 205-211, 1999), and CRAL001 (Okuyama, S. et al., J. Pharmacol. Experimental Therapeut., 289(2), 926-935, 1999), and others known in the art. Each of the above cited documents is hereby incorporated by reference in its entirety.

### CRF Regulatory Antibodies

The invention also contemplates agonistic and antagonistic antibodies. Suitable antagonistic antibodies include those which result in decreased CRF mediated Aβ levels. As such, the antibodies may be specific for CRF protein, transcriptional repressors or activators of CRF, or other proteins required for proper CRF activity. The antagonistic antibodies may be any antibody-like molecule that has an antigen binding region such as monoclonal and polyclonal antibodies, and includes antibody fragments such as Fab′, Fab, F(ab′)₂, single domain antibodies, Fv, single chain Fv, and the like. Further, the antibodies may be anti-idiotypic antibodies that mimic the CRF protein, specifically the CRF receptor binding motif. For example, an antibody specific for the CRF receptor binding motif of CRF is created and then a second antibody specific for the idiotype of the first antibody is created. Similar to a mirror image of a mirror image, the binding site of the anti-idiotype antibody may be an analog of the original antigen. The CRF anti-idiotypic antibody may be capable of binding CRF receptors without subsequent activation. The techniques for preparing and using various antibodies and antibody-based constructs and fragments are well known in the art (Harlow et al., 1988; and U.S. Pat. No. 4,196,265 each incorporated by reference).

### CRF Regulatory Auto-Vaccination

Further, the invention contemplates the use of auto-vaccination technology for generating a strong immune response against otherwise non-immunogenic self-proteins such as CRF to reduce CRF protein activity. For example, potentially self-reactive B-lymphocytes that are able to recognize self-proteins are present in normal individuals. However, in order for these B-lymphocytes to be induced to actually produce antibodies reactive with the relevant self-proteins, assistance is generally needed from cytokine producing T-helper lymphocytes. Normally, this help is not provided because T-lymphocytes, in general, do not recognize T-cell epitopes derived from self-proteins when presented by antigen presenting cells (APCs). By providing an element of “foreignness” in a self-protein (i.e., by introducing an immunologically significant modification), T-cells recognizing the foreign element may be activated upon recognizing the foreign epitope on an APC. Polyclonal B-lymphocytes (which are also APCs) capable of recognizing self-epitopes on the modified self-protein may internalize the antigen and subsequently present the foreign T-cell epitope(s) thereof, and the activated T-lymphocytes subsequently provide cytokine help to these self-reactive polyclonal B-lymphocytes. Since the antibodies produced by these polyclonal B-lymphocytes may be reactive with different epitopes on the modified polypeptide, including those that are also present in the native polypeptide, an antibody cross-reactive with the non-modified self-protein may be induced. The T-lymphocytes may act as if the population of polyclonal B-lymphocytes have recognized an entirely foreign antigen, whereas in fact only the inserted epitope(s) is/are foreign to the host. In this way, antibodies capable of cross-reacting with non-modified self-antigens may be induced.

Methods of modifying a peptide self-antigen in order to obtain breaking of auto-tolerance are known in the art, and are described in U.S. Pat. Application 20020187157, which is incorporated herein by reference. Exemplary modifications include introducing at least one foreign T-cell epitope to the CRF peptide sequence, a moiety that affects targeting of the modified molecule to an APC, a moiety that stimulates the immune system, or a moiety that optimizes presentation of the modified CRF polypeptide to the immune system. Methods of synthesizing modified peptides, and introducing amino acid substitutions are commonly known in the art (see, e.g., Current Protocols in Protein Science, Units 5, pub. John Wiley & Sons, Inc., 2002 and Current Protocols in Protein Science, Units 6, pub. John Wiley & Sons, Inc., 2002, both of which are incorporated herein by reference).

### CRF Regulators

Exemplary CRF agonists that modulate CRF protein activity directly or indirectly include, but are not limited to, synthetic or natural CRF-like peptides and analogs such as fish CRF (Lederis et al. Fish Physiology, Academic Press, San Diego, 1994), urotensis (U.S. Pat. Nos. 4,908,352 and 4,533,654), sauvagine (Erspamer et al. Regulatory Peptides 2:1-13, 1981), α-helical CRF (U.S. Pat. No. 4,594,329), D-isomer CRF analogs (U.S. Pat. No. 5,278,146), synthetic CRF (U.S. Pat. No. 4,489,163), bipotent cyclic CRF analogs (U.S. Pat. No. 5,493,006, WO 96/18649), truncated CRF coding sequence (CRF 4-41), urocortin and urocortin analogs (U.S. Pat. No. 6,214,797), r/h CRF (U.S. Pat. No. 4,489,163), as well as agonists described in U.S. Pat. Nos. 6,326,463, 5,844,074, and 5,824,771, and others known in the art.

Exemplary CRF antagonists that modulate CRF protein activity directly or indirectly include, but are not limited to, synthetic or natural bipotency cyclic CRF antagonists (U.S. Pat. Nos. 5,493,006 and 5,510,458), truncated CRF such as CRF 9-41, CRF 10-41, and CRF8-41, urocortin antagonists and antibodies (U.S. Pat. No. 6,214,797), as well as antagonists described in U.S. Pat. Nos. 6,323,312, 5,777,073, 5,874,227, and others known in the art. Each of the above cited patents or non-patent articles is incorporated herein by reference in its entirety.

### Pharmaceutical Compositions

Therapeutic agents, such as any of the CRF regulators described above or otherwise known in the art, utilized in the present invention for treating AD or other disorders mediated by amyloid-beta accumulation, may be in the form of free bases or pharmaceutically acceptable acid addition salts thereof. The term “pharmaceutically-acceptable salts” embraces salts commonly used to form alkali metal salts and to form addition salts of free acids or free bases. The nature of the salt may vary, provided that it is pharmaceutically acceptable. Suitable pharmaceutically acceptable acid addition salts of compounds of use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound.

As will be appreciated by the skilled artisan, the therapeutic agents of the present invention may be formulated into pharmaceutical compositions and administered by a number of different means that will deliver a therapeutically effective dose. They may be administered locally or systemically. Such compositions may be administered orally, parenterally, by inhalation spray, intrapulmonary, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intraarterial, intraperitoneal, intracochlear, or intrasternal injection, or infusion techniques. The therapeutic agents of the present invention may be administered by daily subcutaneous injection or by implants. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

### Dosage

In general, the dosage of administered CRF regulators containing compounds will vary depending upon such factors as the recipient's age, weight, height, sex, general medical condition and previous medical history. It is preferred that the amount of CRF regulator administered results in altered CRF protein activity and, more preferably, modulation of Aβ levels. CRF protein activity and Aβ levels may be measured by methods described in the Examples herein. Range finding studies may be conducted to determine appropriate dosage by techniques known to those skilled in the art and as described in Current Protocols in Pharmacology, Unit 10, pub. John Wiley & Sons, 2003; Goodman & Goldman's The Pharmacological Basis of Therapeutics, Ninth Edition (1996), Appendix II, pp. 1707-1711; and Goodman & Goldman's The Pharmacological Basis of Therapeutics, Tenth Edition (2001), Appendix II, pp. 475-493, all incorporated herein by reference. A skilled artisan will recognize the effective amount for each CRF regulator may vary with factors including, but not limited to, the activity of the regulator used, stability of the active regulator in the recipient's body, the total weight of the recipient treated, the route of administration, the ease of absorption, distribution, and excretion of the active regulator by the recipient, the age and sensitivity of the recipient to be treated, the type of tissue, and the like.

### Subjects

The methods and compositions of the present invention may be utilized for any mammalian subject. Such mammalian subjects include, but are not limited to, humans and companion animals. Exemplary companion animals may include domesticated mammals (e.g., dogs, cats, horses), mammals with significant commercial value (e.g., dairy cows, beef cattle, sporting animals), mammals with significant scientific value (e.g., captive or free specimens of endangered species), or mammals which otherwise have value.

### DEFINITIONS

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rlgG). See also, Pierce Catalog and Handbook, 1994-1995 (Pierce Chemical Co., Rockford, Ill.). See also, e.g., Kuby, J., Immunology, 3^{rd} Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al., (1992) J Immunol 148:1547, Pack and Pluckthun (1992) Biochemistry 31:1579, Hollinger et al., 1993, supra, Gruber et al. (1994) J Immunol:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

As used herein, Alzheimer's disease (AD) refers to dementia, disease, or disorders associated with the accumulation of Aβ in the parenchyma of the brain and/or in the cerebral arterioles in the form of cerebral amyloid angiopathy (CAA).

As used herein, Aβ refers to a fragment of amyloid precursor protein. Aβ is also referred to as beta-amyloid protein or amyloid-beta protein.

As used herein, “association” refers to the specific binding between two or more molecules. For instance, in reference to a receptor, association encompasses the binding of a ligand to a receptor. Likewise, association encompasses the binding of an antibody to a specific antigen, antisense molecule to the complementary sense molecule, a transcription factor to DNA, a protein to another protein, and other binding situations known to occur in the art.

An “effective amount” is a therapeutically-effective amount that is intended to qualify the amount of an agent or compound, that when administered to a subject, will achieve the goal of preventing, delaying, or treating the cognitive loss associated with dementia due to AD or other disorders mediated by amyloid-beta accumulation.

The terms “modulate,” “modulating,” and “altering,” as used herein, are used in their broadest interpretation and refer to a change in the biological activity of a biologically active molecule. Modulation, or altering, may be an increase or a decrease in activity, a change in binding characteristics, or any other change in the biological, functional, or immunological properties of biologically active molecules. In an exemplary embodiment, “modulation of CRF activity” refers to a change in CRF mediated Aβ levels.

The terms “treat,” “treating,” or “treatment,” as used herein in the context of AD or other disorders mediated by Aβ accumulation, include preventing the damage before it occurs, or reducing loss or damage after it occurs.

As various changes could be made in the above compositions and methods without departing from the scope of the invention, it is intended that all matter contained in the above description and in the examples given below, shall be interpreted as illustrative and not in a limiting sense.

### EXAMPLES

The following examples illustrate various embodiments of the invention.

### Materials and Methods

Animals. All experimental procedures involving animals were performed in accordance with guidelines established by the Animal Studies Committee at Washington University. Tg2576^{+/-} hemizygous male mice (a generous gift from Dr. K. Ashe, University of Minnesota) were bred to C57Bl6/SJL female mice (Taconic Farms, Germantown, N.Y.). The Tg2576^{+/-} littermates of both sexes were used equally for the experimental groups. Animals were screened for the Tg2576 transgene by PCR using DNA obtained from post-weaning toe biopsies. Animals were raised and all experiments were performed in 12 hr dark and 12 hr light controlled rooms. The animals had access to food and water ad lib.

Isolation and restraint stress. To induce chronic isolation stress, Tg2576 mice were individually housed in cages one third the size of a standard mouse cage from weaning until 4 months of age (Dong, H., et al. Neuroscience 127:601-609, 2004; Bartolomucci, A., et al, Psychoneuroendocrinology 28:540-558, 2003). The control animals were group-housed (n=2-5 per standard-sized cage). All mice received food and water ad lib. For restraint stress, mice at 3-4 months of age were subjected to 3 hours of restraint in a 50 ml polypropylene tube (4×5×4 cm) similar to a method described in Chen, Y., et al. Mol Psychiatry 11:992-1002, 2006. The stress was initiated at the beginning of dark period during microdialysis. Mice subjected to restraint were raised under standard group-housing conditions until stress was given. The control animals were subjected to only microdialysis without additional stress.

In vivo microdialysis. In vivo microdialysis to assess brain ISF Aβ₁₋ₓ in the hippocampus of awake, freely moving Tg2576 mice was performed in a manner similar to that previously described Cirrito, J. R., et al. Neuron 48:913-922, 2005, and Cirrito, J. R., et al. J. Neurosci. 23:8844-8853, 2003. This technique samples soluble molecules within the extracellular fluid that are smaller than 38 kDa, the molecular weight cutoff of the microdialysis probe membrane. Briefly, under isoflurane volatile anesthetic, guide cannuli (BR-style, Bioanalytical Systems, Indianapolis, Ind.) were cemented into the left hippocampus (bregma −3.1 mm, 2.5 mm lateral to midline, and 1.2 mm below the dura at a 12° angle). Two millimeter microdialysis probes were inserted through the guide cannula so that the membrane was contained entirely within the hippocampus (BR-2 style probe, 38 kDa MWCO membrane, Bioanalytical Systems). Following probe insertion, mice were permitted to awaken and remained awake for the duration of the experiment. During microdialysis, all mice were housed in RaTurn caging systems (Bioanalytical Systems), which permitted freedom of movement and ad lib food and water. Microdialysis perfusion buffer was artificial CSF (aCSF) containing 0.15% bovine serum albumin that was filtered through a 0.1 μm membrane. Flow rate was a constant 1.5 μl/min. Samples were collected every 60 min with a refrigerated fraction collector into polypropylene tubes and assessed for Aβ₁₋ₓ by ELISA at the completion of each experiment. Basal levels of ISF Aβ were defined as the mean concentration of Aβ from hours 5-10 after probe insertion. In all data from microdialysis experiments, time 1 indicated one hour after the beginning of the dark period unless specifically noted. Following each experiment, animals were sacrificed.

Aβ, apoE, and CRF quantification. Microdialysis samples and hippocampal tissue lysates were analyzed for Aβ using a denaturing, sandwich ELISA specific for human Aβ₁₋ₓ, Aβ₁₋₄₀, or Aβ₁₋₄₂ as described by Cirrito, J. R., et al. J. Neurosci. 23:8844-8853, 2003. Free CRF levels from microdialysis samples were analyzed using a sandwich ELISA kit (COSMO BIO Co., Japan). ApoE levels were assessed in tissue lysates as described by Wahrle, S. E., et al. J Biol Chem 279:40987-93, 2004.

Western Blots. Hippocampal tissues were harvested at the end of 3 months of isolation stress and control conditions or at 14 hr after the beginning of 3 hr of restraint stress initiation and control conditions. Western blots were performed as described by Cirrito, J. R., et al. J. Neurosci. 23:8844-8853, 2003. Briefly, hippocampal tissue from 3-4 month old Tg2576 mice was homogenized in RIPA buffer containing the following: 150 mM NaCl, 50 mM Tris (pH 7.4), 0.5% deoxycholic acid, 0.1% SDS, 1% Triton X-100, 2.5 mM EDTA, and protease inhibitors. Western blotting for full-length APP (FL-APP) and APP-CTF was performed using 4-12% Bis-Tris NuPAGE gels (Invitrogen, Carlsbad, Calif.) under reducing conditions with 30 μg of protein loaded per lane. Nitrocellulose blots were probed with rabbit-anti-APP directed against the C-terminus of APP (Invitrogen), followed by goat anti-rabbit conjugated to peroxidase (BioRad, Hercules, Calif.). The same membrane, cut around 50-60 kDa was probed with rabbit anti-tubulin (Sigma-Aldrich, St. Louis, Mo.) as a loading control protein. Bands were detected with Lumigen-TMA6 (Amersham, Piscataway, N.J.) for APP-CTF or SuperSignal West Pico Chemiluminescence (Pierce) for FL-APP and tubulin. Images were captured digitally using the Kodak ImageStation 440CF. Densitometry was performed using the Kodak 1D Image Analysis software, and each band was normalized to tubulin signal in each lane.

Drug treatment. Tetrodotoxin (TTX) was purchased from Sigma-Aldrich and dissolved in water at 3.13 mM as a stock solution. TTX was diluted in aCSF to a final concentration of 5 μM immediately prior to the experiments and delivered into the hippocampus via reverse microdialysis. Corticosterone (CORT) was purchased from Sigma-Aldrich and dissolved in 15% of 2-hydroxypropyl-β-cyclodextrin (HPB) at 15 mg/ml. Fifty mg/kg bodyweight of CORT or 15% HPB alone as a vehicle in a 100 μl total volume was injected intraperitoneally into mice. Human/rat CRF peptide (h/r CRF) and α-CRF₉₋₄₁ peptide (a CRF receptor antagonist) were purchased from Bachem (King of Prussia, Pa.). For h/r CRF, 400 ng/μl of a stock solution was prepared in 10 mM acetic acid and diluted in aCSF to final concentrations of 100 and 200 nM. For α-CRF_{9-41,} 3 μg/μl of a stock solution was prepared in 10 mM acetic acid and diluted in aCSF to final concentration of 860 nM. Both h/r CRF and α-CRF₉₋₄₁ were diluted in aCSF immediately prior to the experiments and administered directly into the hippocampus by reverse microdialysis.

Statistical Analysis. Data in figures represent mean±SEM. All statistical analyses were performed using Prism version 4.02 for Windows (GraphPad, San Diego). Statistical analysis was performed using a nonparametric Mann-Whitney t test and was accepted as significant if p≦0.05. Comparisons between two groups were performed using two-way ANOVA with Bonferroni post-test.

### Example 1

### Chronic Isolation Stress Increases ISF Aβ Levels

Sporadic, late onset AD accounts for the majority of cases of AD; however, unlike the familial forms, the etiology remains largely unknown. The only genetic risk factor that influences late-onset AD that is confirmed in multiple studies is one's APOE genotype. Environmental factors such as head trauma and education also appear to influence disease risk. Further, evidence from both humans and animal models has suggested that stress can increase the risk for developing AD, but its influence has remained unknown. The invention demonstrates that stress is directly involved in AD disease progression. Specifically, stress directly increases a pool of Aβ that is important in disease progression.

Chronic isolation accelerates the onset and exacerbates Aβ deposition and amyloid load in the hippocampus and cortex of Tg2576 mice (Dong, H., et al. Neuroscience 127: 601-609, 2004), a transgenic mouse model expressing a mutated form of human APP that causes an autosomal dominant form of early-onset AD in humans (Hsiao, K., et al. Science 274: 99-102, 1996). Because the formation of Aβ-containing plaques within the extracellular space is concentration-dependent, the inventors hypothesized that behavioral stressors may increase ISF Aβ levels early in life, thereby leading to Aβ-aggregation and plaque formation. Tg2576 mice of weaning age (3-4 weeks of age) were subjected to 3 months of isolation stress. This time point was selected to avoid assessing animals in which plaques were already present, as their presence can also alter ISF Aβ levels (Cirrito, J. R., et al. J. Neurosci. 23: 8844-8853, 2003). Isolation consisted of rearing a single mouse in a small cage (approximately ⅓ the size of a standard mouse cage). In previous experiments with Tg2576 mice, this treatment was associated with impairments in contextual memory, decreased neurogenesis, and greater Aβ deposition. In contrast, control littermate Tg2576 mice were reared under standard rodent housing conditions (2-5 mice per standard size cage). Brain Aβ levels were assessed in all mice at 4 months of age, an age prior to Aβ deposition even in stressed mice.

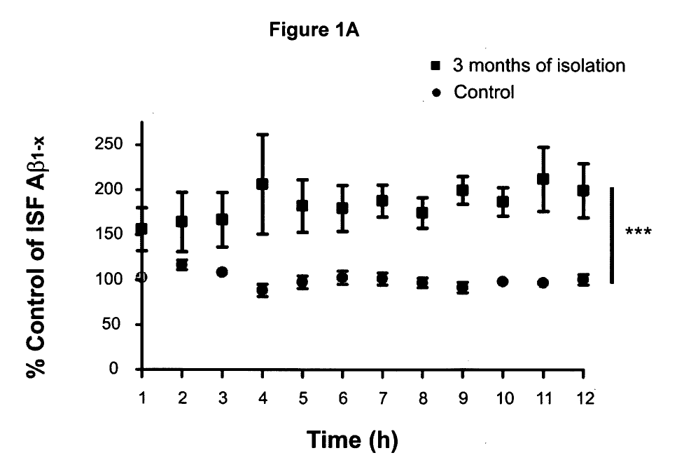

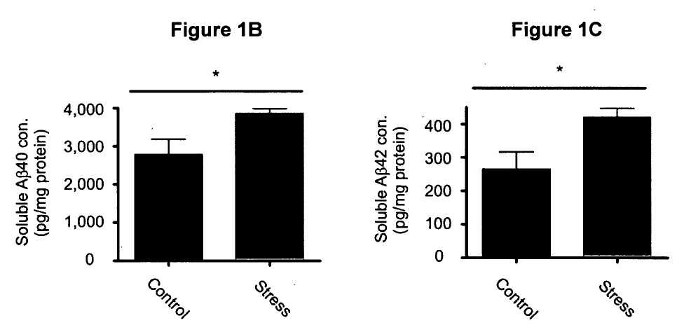

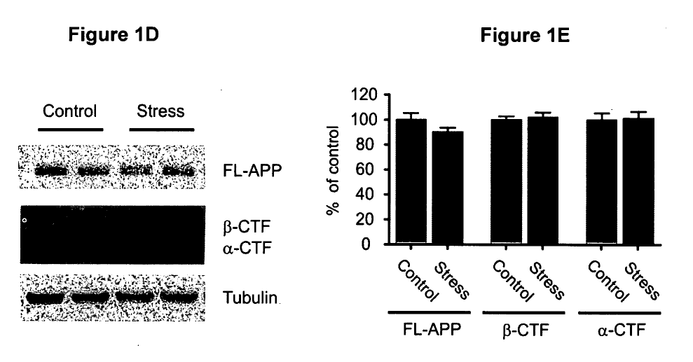

To specifically measure soluble Aβ levels in the brain extracellular space, an in vivo microdialysis technique was utilized to measure ISF Aβ every 60 minutes for 12 hours in awake, behaving mice (Cirrito, J. R., et al. Neuron 48: 913-922, 2005; Cirrito, J. R., et al. J. Neurosci. 23: 8844-8853, 2003). ISF Aβ₁₋ₓ levels were increased by 84% in Tg2576 mice exposed to 3 months of isolation stress, compared to control mice (FIG. 1A). This increase in ISF Aβ levels was likely a key precipitating factor that resulted in accelerated Aβ deposition in Tg2576 mice subjected to 6 months of isolation stress.

The levels of Aβ within hippocampal brain tissue were also assessed in control and chronically isolated Tg2576 mice. Hippocampal tissue was biochemically processed by sequential extraction in carbonate buffer and then 5M guanidine (DeMattos, R. B., et al. Proc Natl Acad Sci USA 99:10843-10848, 2002). Carbonate-soluble Aβ₄₀ and Aβ₄₂ levels were elevated by 38% and 59%, respectively, in 3 month isolated mice compared to controls (FIGS. 1B and 1C). There was not a significant change in the Aβ40/42 ratio in the isolated mice compared to control mice. There were also no significant differences between groups in guanidine soluble Aβ levels, and neither the isolated mice nor the control mice contained Aβ deposition as assessed by immunostaining at this age.

To determine if isolation stress altered APP protein levels or APP processing, the levels of full-length APP, as well as the α- and β-C-terminal fragments (CTF) of APP were assessed using Western blots. Aβ generation from APP requires proteolytic cleavage by β-secretase followed by γ-secretase, thereby producing β- and γ-CTF respectively, whereas cleavage by α-secretase generates α-CTF and precludes Aβ formation. In assessing α-CTF and β-CTF, which serve as markers for APP cleavage, there was no difference in the levels of full-length APP protein, nor was there a difference in α- and β-CTF in mice subjected to 3 months of isolation stress compared to control mice (FIG. 1D). To examine whether isolation stress altered the protein expression levels of Aβ degrading enzymes and apoE, the levels of insulin-degrading enzyme (IDE) and neprilysin (NEP) were assessed in hippocampal tissue by Western blot and the levels apoE were assessed by ELISA. There were no differences in the levels of IDE, NEP, or apoE in mice exposed to 3 months of isolation stress compared to controls.

### Example 2

### Acute Restraint Stress Increases ISF Aβ Levels

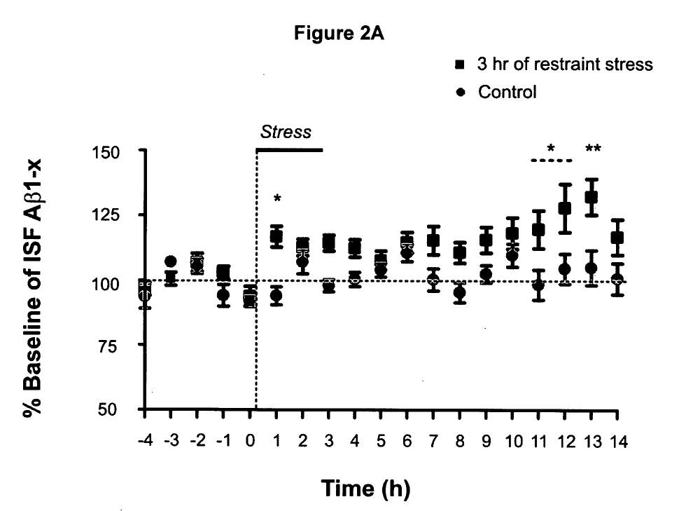

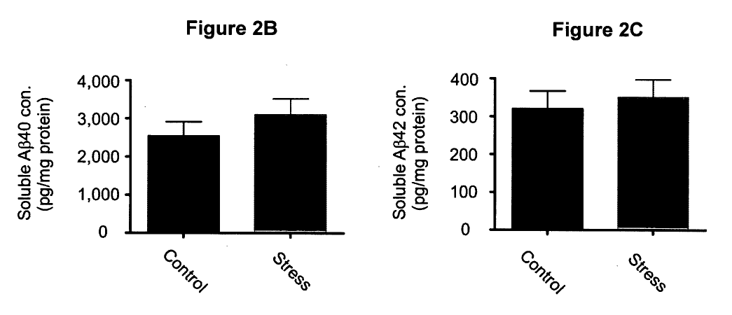

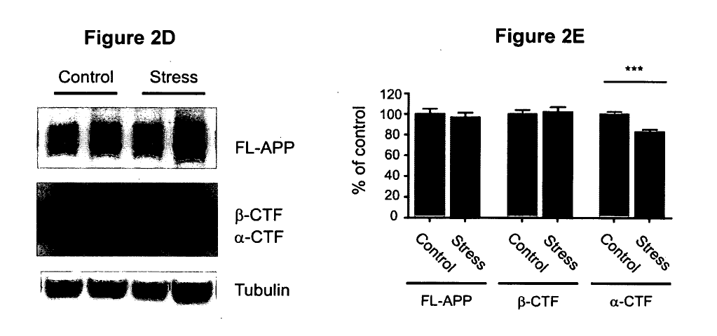

Since chronic stress elevated ISF Aβ, the effect of an acute behavioral stressor on ISF Aβ levels was analyzed. To this end, 3-4 month old Tg2576 (raised under standard housing conditions) were subjected to 3 hours of restraint stress (Harris, R. B., et al. Physiol Behav 73:599-608, 2001). In vivo microdialysis was utilized to assess ISF Aβ levels dynamically prior to, during, and for 11 hours following the end of restraint. Three hours of restraint stress increased ISF Aβ levels within one hour of the initiation of restraint and reached a peak increase of 32% by 13 hours (FIG. 2A). At 13 hours from the beginning of restraint stress, carbonate-soluble Aβ₄₀ and Aβ₄₂ levels were not significantly increased within hippocampal tissue (FIGS. 2B and 2C). Similar to isolation stress, acute restraint stress did not alter the levels of full-length APP or β-CTF in hippocampal tissue at 13 hours from the beginning of restraint (FIG. 2D). Interestingly, there was a small but significant 17% decrease in α-CTF levels in mice subjected to restraint stress (FIG. 2D). Because there was no change in β-CTF, it is unknown if this change in α-CTF is related to the increase in ISF Aβ levels. Given that the decrease in α-CTF is small compared to the 32% increase in ISF Aβ levels, if a change in α-secretase cleavage does contribute to altered Aβ levels, it likely represents a small contribution to the overall effect. Also, the levels of IDE and NEP protein were examined by Western blot and apoE by ELISA in hippocampal tissue 13 hours after the beginning of acute restraint stress. Similar to chronic isolation stress, the levels were not changed in stressed mice compared to controls. The effect on ISF Aβ was greatest when mice were subjected to several months of stress; however, a significant effect of stress could be detected in as little as one hour.

### Example 3

### Acute Corticosterone does not Mimic Stress-Induced Increase in ISF Aβ Levels

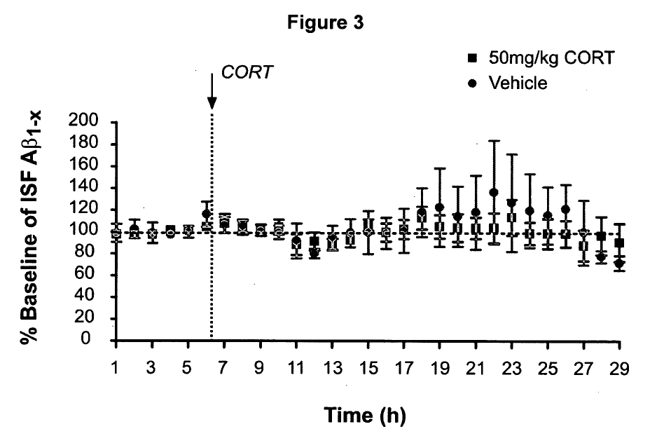

Stressful stimuli activate the hypothalamic-pituitary-adrenal (HPA) axis. One effect of stress is to cause release of corticotropin releasing factor (CRF) from the hypothalamus into the hypophyseal portal system, where it travels to the pituitary gland to cause adrenocorticotropic hormone (ACTH) release, thereby inducing adrenal glucocorticoid release, a major endpoint of the HPA axis. Glucocorticoids act peripherally, as well as within the brain, in response to stressful stimuli. To determine whether systemic administration of corticosterone, the most abundantly produced endogenous glucocorticoid hormone in rodents, could mimic the effect of acute restraint stress on ISF Aβ levels, three to four month old Tg2576 mice were treated with either vehicle or corticosterone (50 mg/kg, intraperitoneally). Basal ISF Aβ levels were measured every hour for 6 hours, as well as an additional 23 hours following treatment. Corticosterone did not alter ISF Aβ levels in Tg2576 as compared to vehicle-treated mice (FIG. 3), suggesting that corticosterone does not mediate acute stress-induced increase in ISF Aβ levels.

### Example 4

### Corticotropin-Releasing Factor (CRF) Mediates the Stress-Induced Increase in ISF Aβ Levels

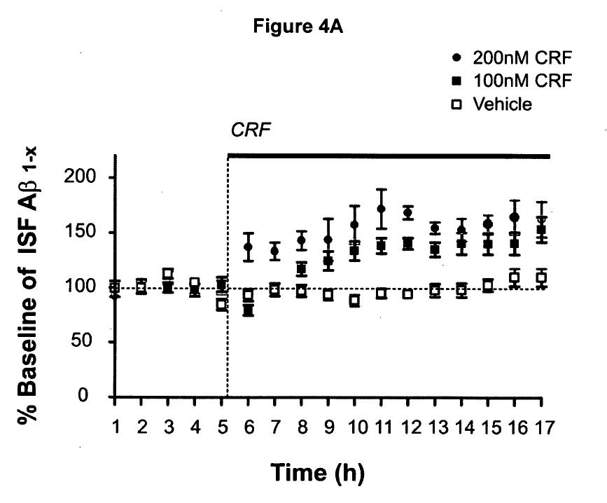

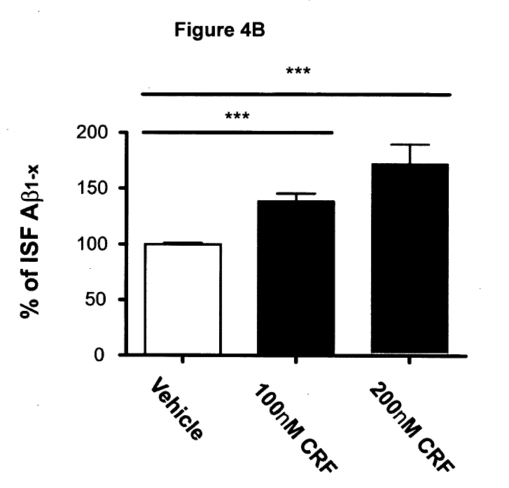

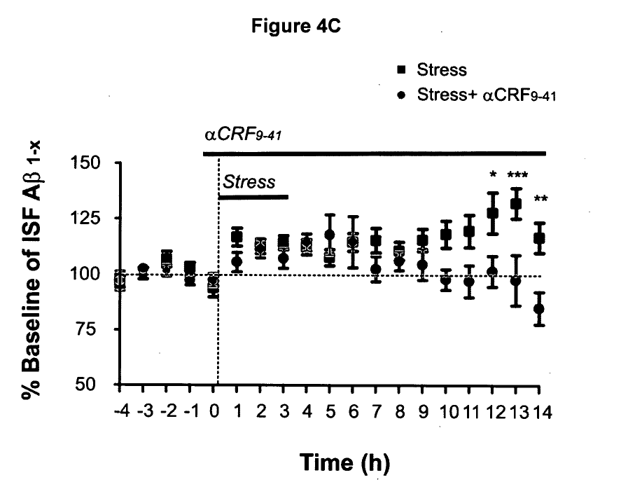

Given that corticosterone is a major hormone in the stress response, it was of interest to determine if a step upstream of corticosterone release contributes to alterations in ISF Aβ levels. In response to stress, CRF peptide is synthesized and released from the hypothalamus to stimulate corticosterone release from the adrenal gland. It is also produced in many brain regions where it may bind to G-protein coupled CRF receptors and facilitate excitatory neurotransmission. In the hippocampus, CRF is synthesized in subsets of hippocampal interneurons. As a response to stress, CRF is released and activates CRF receptors, which are expressed in a majority of CA1 and CA3 pyramidal cells in the hippocampus. Therefore, the ability of CRF to alter the levels of ISF Aβ in the hippocampus was analyzed by infusing CRF directly into the hippocampus by reverse microdialysis. After basal ISF Aβ levels were established in each mouse for 10 hours, the microdialysis perfusion buffer was switched to contain either vehicle or 100 nM or 200 nM CRF. CRF caused an immediate increase in ISF Aβ levels in a dose-dependent manner; 100 and 200 nM CRF increased ISF Aβ levels to 138.3 and 171.9% over 12 hours, respectively (FIGS. 4A and 4B). These data suggest that CRF mediates increases in ISF Aβ levels produced by behavioral stressors.

To further examine if endogenous CRF is responsible for modulating ISF Aβ levels in mice subjected to 3 hours of acute restraint stress, 3 month old Tg2576 mice were pre-treated with either vehicle or αCRF₉₋₄₁, an antagonist of CRF receptors, by reverse microdialysis. αCRF₉₋₄₁ was continuously infused from 30 minutes prior to the onset of 3 hours of restraint stress until the end of the experiment. αCRF₉₋₄₁ prevented the stress-induced increase in ISF Aβ levels (FIG. 4C), suggesting that endogenous CRF likely mediates the increase in ISF Aβ levels caused by behavioral stressors. Infusion with αCRF₉₋₄₁ in the hippocampus, in the absence of stress, had no significant effect on ISF Aβ levels compared to vehicle treated mice. Increases in ISF Aβ levels mediated by endogenous CRF were due to increased endogenous CRF, enhanced sensitivity of CRF receptors, or both.

CRF levels were assessed by ELISA in hippocampal ISF by microdialysis in 3 month old Tg2576 mice subjected to acute restraint stress and chronic isolation stress. After obtaining the basal ISF Aβ levels for 10 hours, 3 hours of restraint stress was given to mice and samples were collected every 3 hours up to 12 hours from the end of restraint. CRF levels were significantly higher in the 3 hour period immediately following 3 hours of acute restraint stress compared to controls (stressed mice, 173.0±24%; control mice, 100.0±15%; mean±SEM; p=0.02; n=5 per each group). This data suggests increases in endogenous CRF may play a role in the acute CRF mediated increase in ISF Aβ levels. CRF levels in the mice exposed to chronic isolation compared to control conditions were also assessed. There was no difference in CRF levels in the mice exposed to 3 months of isolation stress compared to exposure to the control condition (stressed mice, 104.8±12%; control mice, 100.0±19%; data expressed as mean±SEM; n=5 per each group). Collectively, the data suggest that the mechanism of acute vs. chronic stress on ISF Aβ are likely to differ.

### Example 5

### Neuronal/Synaptic Activity is Involved in Stress-Induced Increases in ISF Aβ Levels

Within the hippocampus, CRF potentiates excitatory neurotransmission (Baram, T. Z. and Hatalski, C. G. Trends Neurosci 21:471-476, 1998). Intracellular electrophysiological recordings from rat hippocampal pyramidal neurons determined that exogenously applied CRF increases the firing of CA1 pyramidal neurons in response to excitatory input (Aldenhoff, J. B., et al. Science 221:875-877, 1983). Endogenous CRF during stress also enhances hippocampal synaptic plasticity (Blank, T., et al. J Neurosci. 22:3788-3794, 2002). The inventors previously demonstrated that neuronal and synaptic activity regulates ISF Aβ release from neurons (Cirrito, J. R., et al. Neuron 48:913-922, 2005). Taken together, these studies suggest that the effect of stress on ISF Aβ levels through the actions of CRF and its receptors may result from an increase in excitatory synaptic transmission.

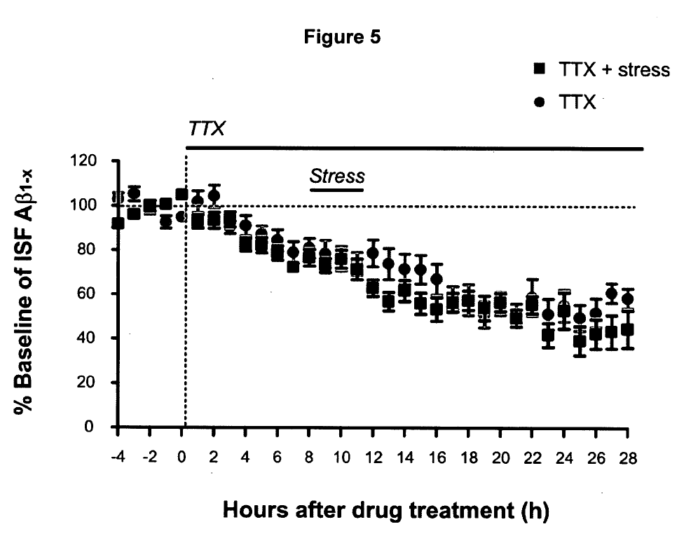

The assessment of this possibility included decreasing the neuronal activity by infusing tetrodotoxin (TTX) directly into the hippocampus by reverse microdialysis. Consistent with the inventors' previous observations (Cirrito, J. R., et al. Neuron 48:913-922, 2005), TTX treatment decreased ISF Aβ levels in Tg2576 mice by ˜60% over 16 hours compared to baseline (FIG. 5). ISF Aβ levels remained low for an additional 12 hours in the presence of TTX. Further, TTX almost completely blocked neuronal activity in the hippocampus by 6 hours of treatment as assessed by extracellular field potential recordings. Therefore, after 8 hours of TTX administration, mice were subjected to three hours of restraint stress. In the presence of TTX, Tg2576 mice subjected to restraint stress had a similar decrease in hippocampal ISF Aβ levels as seen with control mice (FIG. 5). The TTX block of the increase in ISF Aβ levels normally associated with restraint stress suggests that neuronal activity mediates acute stress-induced alterations in ISF Aβ levels. These data are also consistent with findings that neuronal activity is linked to neuronal Aβ release (Cirrito, J. R., et al. Neuron 48:913-922, 2005) and suggests that modulation of ISF Aβ levels through environmental and physiological alterations may result from neuronal activity mediated by specific neuromodulators such as CRF.

In summary, the examples demonstrate that acute and chronic behavioral stressors increase ISF Aβ levels. The acute effects of restraint stress are mediated via effects of CRF and require neuronal activity. The relationship between stress, CRF, and ISF Aβ levels suggest that CRF may play a role in AD pathogenesis and that CRF and CRF signaling pathways are therapeutic targets to modulate processes that affect Aβ metabolism.

What is claimed is:

1. A method for modulating the concentration of amyloid-beta in the brain interstitial fluid of a subject, the method comprising modulating corticotrophin-releasing factor (CRF) activity in the subject, wherein CRF activity modulates the concentration of amyloid-beta.

2. The method of claim 1, wherein modulating CRF activity is mediated by altering a CRF protein production step selected from the group consisting of CRF transcription, CRF translation, and CRF protein activity.

3. The method of claim 1, wherein CRF activity is modulated in the subject by treatment with an effective amount of a CRF regulator.

4. The method of claim 3, wherein the CRF regulator is an antagonist or agonist.

5. The method of claim 3, wherein the CRF regulator is selected from the group consisting of an antibody, a peptide, a protein, a small molecule, an oligonucleotide, RNA antisense, DNA antisense, or combination thereof.

6. The method of claim 3, wherein the CRF regulator decreases CRF activity.

7. The method of claim 3, wherein the concentration of amyloid-beta is decreased.

8. The method of claim 1, wherein CRF activity is modulated by altering CRF association with at least one CRF receptor.

9. The method of claim 8, wherein the CRF receptor is selected from the group consisting of CRF-R1, CRF-R2α, CRF-R2β, and CRF-R2γ.

10. A method for decreasing the concentration of amyloid-beta in the brain interstitial fluid of a subject, the method comprising inhibiting corticotrophin-releasing factor (CRF) activity in the subject.

11. The method of claim 10, wherein inhibiting CRF activity is mediated by repressing or inhibiting a CRF protein production step selected from the group consisting of CRF transcription, CRF translation, and CRF protein activity.

12. The method of claim 10, wherein CRF activity is inhibited in the subject by treatment with an effective amount of a CRF antagonist.

13. The method of claim 12, wherein the CRF antagonist is selected from the group consisting of an antibody, a peptide, a protein, a small molecule, an oligonucleotide, RNA antisense, DNA antisense, or combination thereof.

14. The method of claim 10, wherein CRF activity is inhibited by altering CRF association with at least one CRF receptor.

15. The method of claim 14, wherein the CRF receptor is selected from the group consisting of CRF-R1, CRF-R2α, CRF-R2β, and CRF-R2γ.
